(19) United States
(12) Patent Application Publication
(10) Pub. No.: US 2021/0379233 A1
(43) Pub. Date: Dec. 9, 2021

(54) CLOSE PROXIMITY AIR SANITATION

(71) Applicant: Boski Corporation, Orlando, FL (US)

(72) Inventors: Esmaeil BANAEI, Orlando, FL (US); Justin BOGA, Orlando, FL (US); Andrew MOYLE, Orlando, FL (US)

(21) Appl. No.: 17/397,950

(22) Filed: Aug. 9, 2021

Related U.S. Application Data
(60) Provisional application No. 63/036,861, filed on Jun. 9, 2020.

(51) Int. Cl. A61L 9/20

(52) U.S. Cl. CPC A61L 9/20; A61L 2209/14; A61L 2209/134

(57) ABSTRACT

An air sanitation device may be utilized for sanitizing air particularly in environments where individuals may be in close proximity to one another. The device may include an internal chamber with an air inlet, an air outlet, and an air flow path defined between the inlet and the outlet. The chamber may be pressurized, for instances through an adjustable vacuum. The chamber may include one or more sources of sanitizing radiation, which may be a UV light emitter. Reflective coating may be provided around the internal chamber to improve the coverage of UV light, or other sanitizing radiation. The air flow path may follow a channel whose internal surface may include the reflective coating.

### CROSS REFERENCE TO RELATED APPLICATIONS

This application claims the benefit of U.S. Provisional Application No. 63/036,861 filed Jun. 9, 2020, the disclosure of which is hereby incorporated herein by reference in its entirety.

### FIELD OF THE DISCLOSURE

This disclosure relates generally to air purification devices, and more specifically air purification devices utilizing a sanitizing radiation source such as ultra-violet (UV) light.

### BACKGROUND

Improved air purification units are needed more than ever in view of the 2019 novel corona virus disease (COVID-19) pandemic. The transmission of illnesses caused by airborne pathogens can be greatly diminished using air purification devices, particularly if air purification devices are used in indoor and crowded environments where individuals may be less than 6 feet in proximity from one another. Many such illnesses, such as COVID-19, can be effectively combatted using UV light disinfecting techniques. For example, an area of focus has been utilizing UV C light having a wavelength range between 200 and 280 nm to kill airborne pathogens. While UV light has proven to be an effective disinfecting technique, there is a lack of air purification units that effectively utilize UV light disinfecting techniques in close proximity environments.

### BRIEF SUMMARY OF THE DISCLOSURE

The following presents a simplified summary of the disclosure in order to provide a basic understanding of some aspects of the various embodiments disclosed herein. This summary is not an extensive overview of every detail of every embodiment. It is intended to neither identify key or critical elements of every embodiment nor delineate the scope of every disclosed embodiment. Its sole purpose is to present some concepts of disclosure in a simplified form as a prelude to the more detailed description that is presented later.

In one embodiment of the disclosure, an air sanitation device may include an internal chamber including an air inlet, an air outlet, and an air flow path defined between the air inlet and the air outlet. The device may also include a sanitizing radiation source provided within the internal chamber and positioned to emit sanitizing radiation onto at least a portion of the air flow path.

The following description and annexed drawings set forth certain illustrative aspects of the disclosure. These aspects are indicative, however, of but a few of the various ways in which the principles disclosed may be employed. Other advantages and novel features disclosed herein will become apparent from the following description when considered in conjunction with the drawings.

### BRIEF DESCRIPTION OF THE FIGURES

FIG. 1 illustrates an embodiment of an air sanitization device including a filter.

FIG. 2 illustrates an embodiment of an air sanitization device with a plurality of baffles.

FIG. 3 illustrates an embodiment of an air sanitization device with a plurality of baffles and a plurality of sanitization sources.

FIG. 4 illustrates an embodiment of an air sanitization device with a plurality of baffles and a plurality of reflectors.

FIG. 5 illustrates an embodiment of an air sanitization device with a pressurized chamber.

FIG. 6 illustrates an embodiment of an air sanitization device with a funnel.

### DETAILED DESCRIPTION OF THE DISCLOSURE

The following detailed description and the appended drawings describe and illustrate some embodiments for the purpose of enabling one of ordinary skill in the relevant art to make use the invention. As such, the detailed description and illustration of these embodiments are purely illustrative in nature and are in no way intended to limit the scope of the invention, or its protection, in any manner. It should also be understood that the drawings are not necessarily to scale and in certain instances details may have been omitted, which are not necessary for an understanding of the disclosure, such as details of fabrication and assembly. In the accompanying drawings, like numerals represent like components.

In one embodiment an air sanitation device may include an internal chamber including an air inlet, an air outlet, and an air flow path defined between the air inlet and the air outlet. The device may also include a sanitizing radiation source provided within the internal chamber and positioned to emit sanitizing radiation onto at least a portion of the air flow path.

1. In further embodiments, the device may include a plurality of baffles positioned within the internal chamber, and the air flow path may be defined through the baffles. A plurality of additional sanitizing radiation sources may each be positioned proximate one of the plurality of baffles. A plurality of reflectors may each be positioned proximate one of the plurality of baffles. At least a portion of the air flow may be through a channel having an internal surface formed with, or coated by, reflective material. The internal chamber may be pressurized. The device may include a piston and sleeve along the air flow path, the piston operable to create pressure differentials within sleeve in order to force air along the air flow path. The device may include a vacuum in fluid communication with the air flow path and operable to create a pressure differential in order to force air along the air flow path. The sanitizing radiation source may be a source for emitting UV light. The device may include a filter positioned around the sanitizing radiation source, the filter operable to reduce sanitizing radiation transmitted to at least a portion of the internal chamber outside the filter. The device may include a funnel positioned within the internal chamber proximate the air inlet. The sanitizing radiation source may be aligned with a narrow end of the funnel. At least a portion of the internal chamber may be coated with UV reflective material.

In further embodiments of the disclosure, the device may include a pressure differential device positioned in fluid communication with the airflow path and operable to generate an air flow along the air flow path. The device may also include a channel around at least a portion of the air flow path with an internal surface of the channel coated in UV reflective material. The sanitizing radiation source may emit UV light. The pressure differential device may be a vacuum provided in a sleeve along the air flow path. The device may include a one-way valve at the air inlet. The device may include a pressure valve at the air outlet. The pressure differential device may be adjustable to regulate air flow along the air flow path. The channel may be coiled through the internal chamber

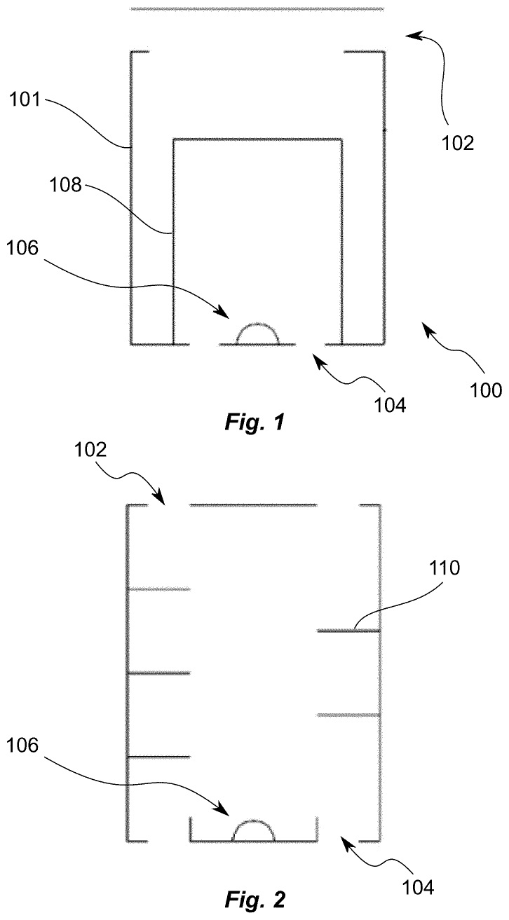

Referring now to FIG. 1, an embodiment of an air sanitation device 100 may include an air chamber 101 having an air inlet 102 and air outlet 104. A UV light source 106 may also be provided within chamber 101. Air may enter chamber 101 through inlet 102 through the assistance of a fan (not illustrated) that may be attached or mounted to chamber 101. Air may then pass through a light barrier or filter 108, which may be a light-colored porous barrier that surrounds UV light source 106. UV light source 106 may be an assembly of multiple light sources, and may be UV lamp on an ultra violet LED. Air may slow down as it passes through barrier 108 thus increasing the air dwell time at the surface of barrier 108. Barrier 108 may have a light color to reduce UV light absorption. Alternatively or additionally, barrier 108 made be composed of polytetrafluoroethylene (PTFE) or similar material with high UV scattering. Barrier 108 may also be coated with reflective materials such as aluminum or silver.

FIG. 2 illustrates an embodiment of an air sanitation device 100 that includes a plurality of barriers or baffles 110. These barriers 110 may be installed within the interior chamber 101 in order to create turbulent air flow within chamber 101. A turbulent air flow may force the air to stay within chamber 101 for a longer period of time, thereby increasing the exposure time to UV light from light source 106 and improving disinfection. As illustrated, barriers 110 may be placed between inlet 102 and outlet 104, and barriers may be dimensioned to prevent blocking the air exposure to light source 106.

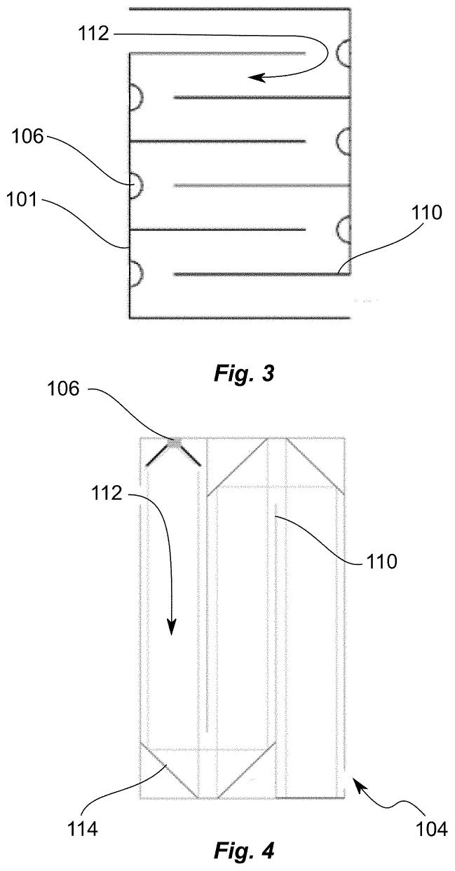

FIGS. 3 and 4 illustrate embodiments of an air sanitation device 100 that includes a series of baffles 110 defining an air flow path 112 through the interior of chamber 101 beginning at an inlet 102 and ending at an outlet 104. Baffles 110 may be arranged so as to increase the length of path 112 through chamber 101. A plurality of UV sources 106 may be implemented, and a UV source 106 may be positioned at each turn along path 112 thereby ensuring each portion of path 112 is exposed to UV light. In another embodiment, a single UV source 106 maybe provided a series of reflectors 114 may be placed at the ends of the baffles 110 to ensure each portion of path 112 is exposed to UV light. For example, as illustrated, a cone reflector 114 may be positioned around a UV source 106, and a pair of angled reflectors 114 may be provided at curve to path 112 with a flat reflector 114 placed at the end of path 112 proximate outlet 104. Additional fans (not illustrated) may be installed between baffle 110 layers to further improve the air flow along path 112.

In some embodiments, a sanitizing radiation source may be utilized in addition to, or in lieu of, UV light source 106. Such ionizing radiation sources may include blue/violet visible light, ultraviolet, x-ray, gamma radiation, atomic-decay particles or other ionizing radiation as a source of sanitization. Non-ionizing radiation may be used such as microwave.

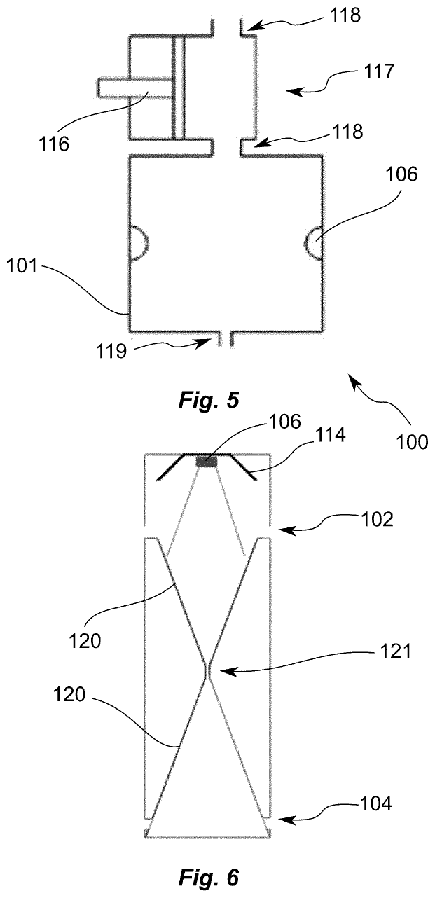

FIG. 5 illustrate an embodiment of air sanitation device 100 where chamber 101 is pressurized at a pressurization level above atmospheric pressure for at least a period of time to improve disinfection of air within chamber 101. In some embodiments, a piston 116 operating in a sleeve 117 may be provided along with one or more valves 118. Sleeve 117 may be portion of chamber 101, which in the illustrated embodiment is proximate inlet 102. An inlet valve 118 may be provided proximate air inlet 102 while an outlet valve 118 may be provided proximate the remainder of chamber 101. Valves 118 may be timed with the actuation of piston 116 to create pressure differentials to suck air in through inlet 102 and expel the air into the remainder of chamber 101 where UV sources 106 are positioned. Sleeve valves 118 may be one valves to ensure a unilateral direction of air flow through sleeve 117. An additional pressure valve 119 may be provided proximate air outlet 104. Pressure valve 119 may open upon reaching a certain pressure, or alternatively the valve may be timed to open after preset number of piston 116 revolutions. Additional pistons 116 time in unison may be provided to further improve air flow. In lieu of, or in addition to, one or more pistons 116, a vacuum may be utilized to forcibly extract air from the atmosphere into internal chamber 101. Vacuums, pistons, or other pressure generating devices may operate to forcibly push air along air flow path, and these pressure generating devices be tunable to adjust the pressure, and consequentially flow force, of the air along the air flow path 112.

FIG. 6 illustrates an embodiment where chamber 101 has two funnel sections 120, having a conical or triangular cross-section, with the two funnels 120 joined by a nozzle 121. The gradual change in dimensions of funnels 120 may promote adiabatic air flow towards nozzle 121. Inlet side and outlet side funnels 120 may have identical or similar dimensions such that the cross-sectional change is gradual as air moves towards or away from the center nozzle 121. In further embodiments, outlet side funnel 120 may be wide open particularly to allow more turbulent air flow. In the illustrated embodiment, the UV light source 106 is shown proximate the air inlets and the inlet side funnel 120. UV light source 106 is also aligned with nozzle 121 so as to permit UV light to pass to the outlet side funnel. Reflectors 114 may also be utilized, as more fully detailed with respect to other embodiments described in this disclosure. A conic reflector 114 is shown in the illustrated embodiment.

In some embodiments, chamber 101 may be formed from, or coated with, a reflective or scattering material to promote expansive coverage of UV light. This may ensure UV light is evenly distributed within chamber 101.

In further embodiments, both light and air may travel through a hollow channel that functions as both an air channel and a light waveguide. This dual purpose channel can be a flexible or rigid tube, hose or channel with interior walls coated with a metallic refractor material or light scattering material such as PTFE. Fans may be mounted directly at the inlet of the channel and a UV light source may be mounted at a point after the fan on the side of the channel either parallel to the channel or slightly angled as may be necessary to optimize the air flow path. Depending on the dwell time needed to adequately sanitize the air, the length of channel can be adjusted. Longer channels may require more UV sources. Multiple channels may be utilized within a single chamber, with each receiving its own fan and UV source, or in some embodiments the fan, UV source, or both being shared between the various channels. In one embodiment, two channels may be provided and eventually joined in a “Y” split to serve multiple inlets but only one outlet.

Various embodiments utilize fans to propagate air flow. It should be appreciated that fans may be strategically placed within, or in fluid connection with, the interior chamber to direct air flow where greater UV light coverage exists. For example, cost restraints may restrict the number of UV light sources that can be utilized, however directed air flow by strategically positioning fans near the air flow path can nevertheless ensure the air has ample exposure to UV light.

To facilitate a compact design, multiple channels can be rolled or wrapped around a core in a spiral form. Similarly, a three-dimensional stack of channels may be utilized to maximize space of the internal chamber. A stacking design may serve to lengthen a channel, which could increase air dwell time within UV light due to the greater area of exposure as well as the larger air pressure drop to the air flow path.

Cross-sectional dimensions shown in the figures are representative examples of possible dimensions. In three dimensions, the device may be circular, square, oval, rectangular, or have other channel or internal chamber dimensions. Additionally, the sizes may be variable such that miniaturized versions of various embodiments may be adapted as wearable air sanitation devices, with the device portable and even mountable on a user's clothing.

The descriptions set forth above are meant to be illustrative and not limiting. Various modifications to the disclosed embodiments, in addition to those described herein, will be apparent to those skilled in the art from the foregoing description. Such modifications are also intended to fall within the scope of the concepts described herein. The disclosures of each patent, patent application, and publication cited or described in this document are hereby incorporated herein by reference, in their entireties.

The foregoing description of possible implementations consistent with the present disclosure does not represent a comprehensive list of all such implementations or all variations of the implementations described. The description of some implementations should not be construed as an intent to exclude other implementations described. For example, artisans will understand how to implement the disclosed embodiments in many other ways, using equivalents and alternatives that do not depart from the scope of the disclosure. Moreover, unless indicated to the contrary in the preceding description, no particular component described in the implementations is essential to the invention. It is thus intended that the embodiments disclosed in the specification be considered illustrative, with a true scope and spirit of invention being indicated by the following claims.

What is claimed:

1. An air sanitation device comprising:
an internal chamber including an air inlet, an air outlet, and an air flow path defined between the air inlet and the air outlet; and
a sanitizing radiation source provided within the internal chamber and positioned to emit sanitizing radiation onto at least a portion of the air flow path.

2. The device of claim 1 further comprising a plurality of baffles positioned within the internal chamber, and the air flow path is defined through the baffles.

3. The device of claim 2 further comprising a plurality of additional sanitizing radiation sources each positioned proximate one of the plurality of baffles.

4. The device of claim 2 further comprising a plurality of reflectors each positioned proximate one of the plurality of baffles.

5. The device of claim 1, wherein at least a portion of the air flow is through a channel having an internal surface formed with, or coated by, reflective material.

6. The device of claim 1, wherein the internal chamber is pressurized.

7. The device of claim 6 further comprising a piston and sleeve along the air flow path, the piston operable to create pressure differentials within sleeve in order to force air along the air flow path.

8. The device of claim 6 further comprising a vacuum in fluid communication with the air flow path and operable to create a pressure differential in order to force air along the air flow path.

9. The device of claim 1 wherein the sanitizing radiation source is a source for emitting UV light.

10. The device of claim 1 further comprising a filter positioned around the sanitizing radiation source, the filter operable to reduce sanitizing radiation transmitted to at least a portion of the internal chamber outside the filter.

11. The device of claim 1 further comprising a funnel positioned within the internal chamber proximate the air inlet.

12. The device of claim 11, wherein the sanitizing radiation source is aligned with a narrow end of the funnel.

13. The device of claim 1 wherein at least a portion of the internal chamber is coated with UV reflective material.

14. The device of 1 further comprising:
a pressure differential device positioned in fluid communication with the airflow path and operable to generate an air flow along the air flow path; and
a channel around at least a portion of the air flow path with an internal surface of the channel coated in UV reflective material,
wherein the sanitizing radiation source emits UV light.

15. The device of claim 14 wherein the pressure differential device is a vacuum provided in a sleeve along the air flow path.

16. The device of claim 15 further comprising a one-way valve at the air inlet.

17. The device of claim 16 further comprising a pressure valve at the air outlet.

18. The device of claim 14 wherein the pressure differential device is adjustable to regulate air flow along the air flow path.

19. The device of claim 14 wherein the channel is coiled through the internal chamber.
